(19) United States
(12) Patent Application Publication
(10) Pub. No.: US 2026/0315792 A1
(43) Pub. Date: Oct. 15, 2026

(54) SYSTEM FOR ELECTROPORATION OF CANCEROUS TUMORS

(71) Applicant: Mohammad Abdolahad, Tehran (IR)

(72) Inventors: Mohammad Abdolahad, Tehran (IR); Ebrahim Karimi, Tehran (IR); Ebrahim Kousha, Tehran (IR); Farshid Rostami Pouria, Tehran (IR); Navid Manoochehri, Tehran (IR); Seyed Mojtaba Yazdanparast, Tehran (IR); Hamed Abadijoo, Tehran (IR); Farrokh Heidari, Tehran (IR)

(73) Assignee: Mohammad Abdolahad, Tehran (IR)

(21) Appl. No.: 19/426,138

(22) Filed: Dec. 19, 2025

Related U.S. Application Data
(63) Continuation-in-part of application No. PCT/IB2025/053860, filed on Apr. 13, 2025.

(51) Int. Cl. A61N 1/36; A61N 1/05

(52) U.S. Cl. CPC A61N 1/36002; A61N 1/0502; A61N 1/36017; A61N 1/36034

(57) ABSTRACT

A system for electroporation of cancerous tumors includes a pair of electrodes and an electric pulse generator. The pair of electrodes delivers a sequence of electric pulses to a cancerous tumor. Each electrode includes a body, a fixed needle, and a replaceable needle. The body is made of a conductive material and is covered with a biocompatible insulator. The fixed needle is made of a conductive material and is attached to a tip of the insertion portion. The replaceable needle is made of a conductive material and is attached to a tip of the fixed needle. The electric pulse generator is electrically coupled to the pair of electrodes and generates the sequence of electric pulses.

### CROSS-REFERENCE TO RELATED APPLICATIONS

This application is a Bypass Application to PCT Application No. PCT/IB2025/053860, filed Apr. 13, 2025, which is incorporated herein by reference in its entirety.

### TECHNICAL FIELD

The present disclosure generally relates to electrochemotherapy, and particularly, to electroporation.

### BACKGROUND ART

Electrochemotherapy is an emerging and effective treatment that utilizes electroporation to increase uptake of chemotherapy in cancerous cells. However, electrochemotherapy has mostly been used in treatment of cutaneous and subcutaneous tumors as these are easily accessible for conventional electrodes. Nevertheless, electroporation can be useful for treatment of various cancerous tumors that may appear in different organs throughout a living body. Therefore, there is a need in the art for a device and system having appropriate electrodes with proper structural features to access cancer tumors at different positions inside a living body; thereby, providing a possibility of performing exact and targeted electroporation on such cancerous tumors.

### SUMMARY OF THE DISCLOSURE

This summary is intended to provide an overview of the subject matter of this patent, and is not intended to identify essential elements or key elements of the subject matter, nor is it intended to be used to determine the scope of the claimed implementations. The proper scope of this patent may be ascertained from the claims set forth below in view of the detailed description below and the drawings.

In one general aspect, the present disclosure describes an exemplary system for electroporation of cancerous tumors. An exemplary system may include a pair of electrodes and an electric pulse generator. An exemplary pair of electrodes may allow for delivering a sequence of electric pulses to a cancerous tumor.

In an exemplary embodiment, each respective electrode of the pair of electrodes may include a body, a fixed needle, and a replaceable needle. An exemplary body may be made of a conductive material and may be covered with a biocompatible insulator. An exemplary body may include a grip portion, an insertion portion, and a curved portion between the grip portion and the insertion portion. An exemplary grip portion may allow for being held by an operator of the system. An exemplary insertion portion may allow for being inserted into a patient's body. An exemplary angle between an axis of the grip portion and an axis of the insertion portion may be between about 90 degrees and about 120 degrees. An exemplary length of the insertion portion may be two to three times larger than a length of the grip portion.

In an exemplary embodiment, the fixed needle may be made of a conductive material and may be attached to a tip of the insertion portion. An exemplary replaceable needle may be made of a conductive material and may allow for being attached to a tip of the fixed needle. An exemplary fixed needle may be covered with a biocompatible insulator.

In an exemplary embodiment, the replaceable needle may include a proximal portion and a distal portion. An exemplary proximal portion may be covered with a biocompatible insulator and may allow for being attached to the tip of the fixed needle. An exemplary distal portion may include a tip of the replaceable needle. An exemplary tip of the replaceable needle may allow for being in contact with the cancerous tumor. An exemplary length of the distal portion may be between about 1 cm and about 3 cm. An exemplary replaceable needle may include a hollow tube. An exemplary proximal end of the hollow tube may encompass the tip of the fixed needle.

An exemplary electric pulse generator may be electrically coupled to the pair of electrodes. In an exemplary embodiment, electric pulse generator may include a pair of wires. In an exemplary embodiment, each respective wire of the pair of wires may be electrically coupled to a respective electrode of the pair of electrodes. An exemplary electric pulse generator may allow for generating the sequence of electric pulses. An exemplary sequence of electric pulses may include electric pulses with a magnitude in a range of about 400 V/cm to about 1500 V/cm and a duration in a range of about 90 μs to about 110 μs.

An exemplary system may further include a pair of connectors. In an exemplary embodiment, each respective connector of the pair of connectors may allow for connecting a respective wire of the pair of wires to a respective electrode of the pair of electrodes. An exemplary system may further include an imaging system that may allow for capturing real-time images from the cancerous tumor, surrounding environment of the cancerous tumor, and a path from a respective location of each of the pair of electrodes to the cancerous tumor.

Other exemplary systems, methods, features and advantages of the implementations will be, or will become, apparent to one of ordinary skill in the art upon examination of the following figures and detailed description. It is intended that all such additional systems, methods, features and advantages be included within this description and this summary, be within the scope of the implementations, and be protected by the claims herein.

### BRIEF DESCRIPTION OF DRAWINGS

The drawing figures depict one or more implementations in accord with the present teachings, by way of example only, not by way of limitation. In the figures, like reference numerals refer to the same or similar elements.

FIG. 1 shows a schematic of a system for electroporation of cancerous tumors, consistent with one or more exemplary embodiments of the present disclosure.

FIG. 2 shows a schematic of an electrode, consistent with one or more exemplary embodiments of the present disclosure.

FIG. 3 shows a schematic of an electrode with a fixed needle covered with a biocompatible insulator, consistent with one or more exemplary embodiments of the present disclosure.

FIG. 4 shows a schematic of an electrode with a replaceable needle partially covered with a biocompatible insulator, consistent with one or more exemplary embodiments of the present disclosure.

FIG. 5 shows a schematic of replaceable needle attached to a fixed needle, consistent with one or more exemplary embodiments of the present disclosure.

FIG. 6 shows a high-level functional block diagram of a computer system, consistent with one or more exemplary embodiments of the present disclosure.

### DESCRIPTION OF EMBODIMENTS

In the following detailed description, numerous specific details are set forth by way of examples in order to provide a thorough understanding of the relevant teachings. However, it should be apparent that the present teachings may be practiced without such details. In other instances, well known methods, procedures, components, and/or circuitry have been described at a relatively high-level, without detail, in order to avoid unnecessarily obscuring aspects of the present teachings.

The following detailed description is presented to enable a person skilled in the art to make and use the methods and devices disclosed in exemplary embodiments of the present disclosure. For purposes of explanation, specific nomenclature is set forth to provide a thorough understanding of the present disclosure. However, it will be apparent to one skilled in the art that these specific details are not required to practice the disclosed exemplary embodiments. Descriptions of specific exemplary embodiments are provided only as representative examples. Various modifications to the exemplary implementations will be readily apparent to one skilled in the art, and the general principles defined herein may be applied to other implementations and applications without departing from the scope of the present disclosure. The present disclosure is not intended to be limited to the implementations shown, but is to be accorded the widest possible scope consistent with the principles and features disclosed herein.

Herein is disclosed an exemplary system for electroporation of cancerous tumors. An exemplary system may include a pair of electrodes that may be electrically coupled to an electric pulse generator. An exemplary pair of electrodes may be utilized by an operator to transmit a sequence of electric pulses to the cancerous tumor for preforming an electroporation process. For this purpose, one of an exemplary pair of electrodes may be placed on a fixed point of a surface of the cancerous tumor while another electrode may sweep on the surface.

Each exemplary electrode may include a body, a fixed needle attached to the body, and a replaceable needle attached to the fixed needle. An exemplary body, shielded by a biocompatible insulator, may be held by an operator performing electroporation and the replaceable needle may touch the surface of an exemplary cancerous tumor. An exemplary replaceable needle may be replaced after each instance of electroporation procedure. An exemplary length of the replaceable needle may be chosen based on the location of the cancerous tumor. Longer replaceable needles may be utilized to perform electroporation on deeper tumors in a patient's body. Based on the location of the tumor, exemplary fixed and replaceable needles may be covered by biocompatible insulators to prevent the electrodes from touching undesired objects.

An exemplary system may further include an imaging system (such as an endoscope) that may capture real-time images so that the positions of electrodes may be viewed in real-time by an operator of the system. As a result, an exemplary system may be utilized for performing electroporation on tumors that may be present at different organs inside a patient's body, such as larynx (in the throat), liver, etc.

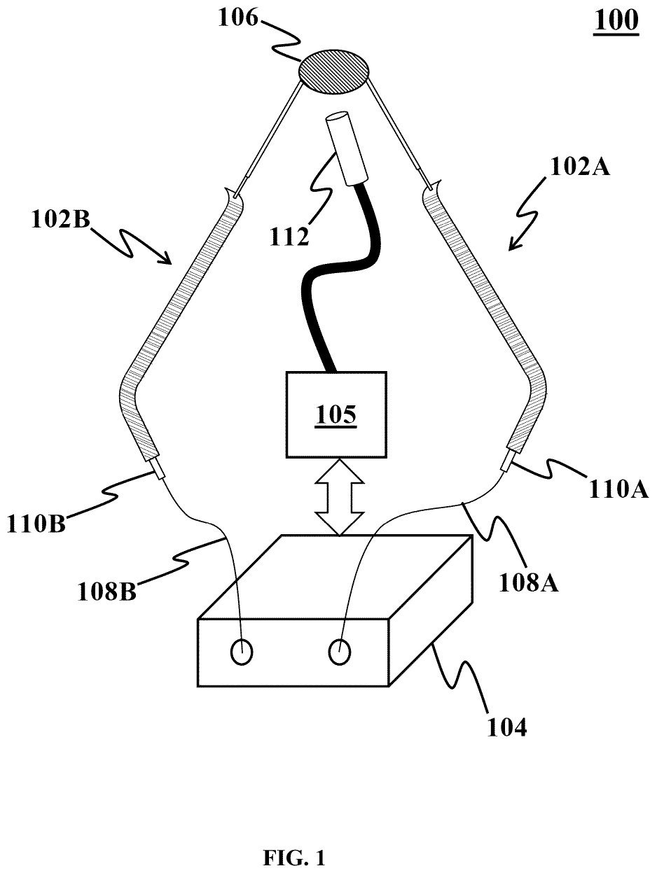

FIG. 1 shows a schematic of a system for electroporation of cancerous tumors, consistent with one or more exemplary embodiments of the present disclosure. As used herein, an exemplary “electroporation” may refer to a process of electrically stimulating living biological cells. In an exemplary embodiment, an electroporation process may include an electrical stimulation of biological cells, leading to changing membrane characteristics of exemplary biological cells; thereby introducing desired molecules such as genetic materials, proteins, drugs, etc. to exemplary biological cells. In an exemplary embodiment, electrical stimulation may include electrical stimulation of cancer cells, leading to at least one of ablation of exemplary cancer cells, destruction of exemplary cancer cells, changing membrane characteristics of exemplary cancer cells; thereby introducing desired molecules such as drugs to exemplary cancer cells. An exemplary system 100 may include a pair of electrodes 102A and 102B, an electric pulse generator 104, and a processor 105. In an exemplary embodiment, pair of electrodes 102A and 102B may allow for delivering a sequence of electric pulses to a cancerous tumor 106, leading to electroporating cancerous tumor 106 and resulting in ablation of cancerous tumor 106.

In an exemplary embodiment, electric pulse generator 104 may be electrically coupled to pair of electrodes 102A and 102B. In an exemplary embodiment, electric pulse generator 104 may include a pair of wires 108A and 108B. In an exemplary embodiment, each respective wire of pair of wires 108A and 108B (for example, wire 108A) may be electrically coupled to a respective electrode (for example, electrode 102A) of pair of electrodes 102A and 102B.

In an exemplary embodiment, system 100 may further include a pair of connectors 110A and 110B. In an exemplary embodiment, each respective connector of pair of connectors 110A and 110B may allow for connecting a respective wire of pair of wires 108A and 108B to a respective electrode of pair of electrodes 102A and 102B. For example, connector 110A may allow for connecting wire 108A to electrode 102A.

In an exemplary embodiment, system 100 may further include an imaging system 112. In an exemplary embodiment, imaging system 112 may allow for capturing real-time images from cancerous tumor 106, surrounding environment of cancerous tumor 106, and a path from a respective location of each of pair of electrodes 102A and 102B to cancerous tumor 106. In an exemplary embodiment, imaging system 112 may include one or more different types of medical imaging devices, such as an endoscope, a magnetic resonance imaging (MRI) scanner, a computed tomography (CT) scanner, an ultrasound scanner, a camera, etc.

In an exemplary embodiment, processor 105 may configure electric pulse generator 104 to generate the sequence of electric pulses. An exemplary sequence of electric pulses may include electric pulses with a magnitude in a range of about 400 V/cm to about 1500 V/cm and a duration in a range of about 90 μs to about 110 μs.

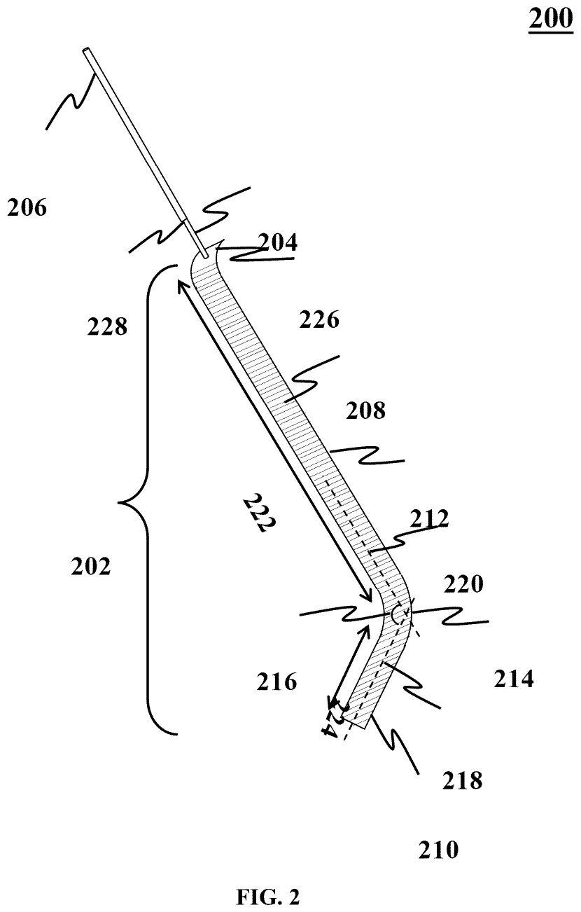

FIG. 2 shows a schematic of an electrode, consistent with one or more exemplary embodiments of the present disclosure. An exemplary electrode 200 may have a similar structure to each electrode of pair of electrodes 102A and 102B (for example, electrode 102A). In an exemplary embodiment, electrode 200 may include a body 202, a fixed needle 204, and a replaceable needle 206. In an exemplary embodiment, body 202 may be made of a conductive material (for example, a metal) and may be covered with a biocompatible insulator 208. In an exemplary embodiment, biocompatible insulator 208 may be made of at least one of polypyrrole, polylactic acid (PLA), silicone rubber, polyethylene terephthalate (PET), polydimethylsiloxane (PDMS), polyethylene, and combinations thereof. In an exemplary embodiment, body 202 may include a grip portion 210, an insertion portion 212, and a curved portion 214 between grip portion 210 and insertion portion 212. In an exemplary embodiment, grip portion 210 may allow for being held by an operator of system 100. In an exemplary embodiment, insertion portion 212 may allow for being inserted into a patient's body. An exemplary angle 216 between an axis 218 of grip portion 210 and an axis 220 of insertion portion 212 may be between about 90 degrees and about 120 degrees to facilitate holding electrode 200 by an operator. An exemplary length 222 of insertion portion 212 may be about two to three times larger than a length 224 of grip portion 210. In an exemplary embodiment, length 222 may be determined based on a depth of cancerous tumor 106 inside a patient's body. Therefore, length 222 may be different in different applications of electrochemotherapy.

In an exemplary embodiment, fixed needle 204 may be made of a conductive material (for example, a metal) and may be attached to a tip 226 of insertion portion 212. In an exemplary embodiment, replaceable needle 206 may be made of a conductive material (for example, a metal) and may allow for being attached to a tip 228 of fixed needle 204. In an exemplary embodiment, body 202, fixed needle 204 and replaceable needle 206 may be made of electrically conductive materials to allow for passing electrical signals from electric pulse generator 104 to cancerous tumor 106.

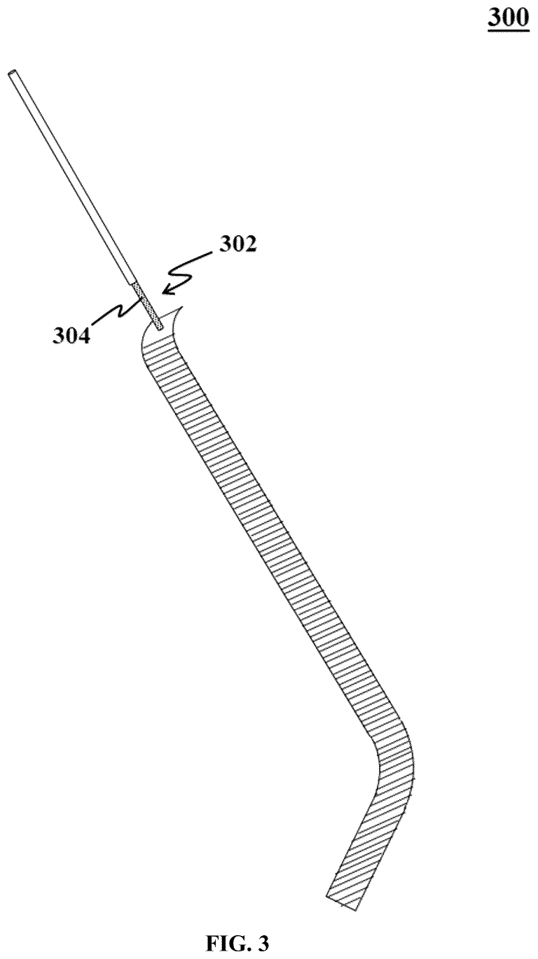

FIG. 3 shows a schematic of an electrode with a fixed needle covered with a biocompatible insulator, consistent with one or more exemplary embodiments of the present disclosure. Referring to FIG. 2 and FIG. 3, an exemplary electrode 300 may have a similar structure to electrode 200 and may further include an exemplary fixed needle 302 (similar to fixed needle 204) that may be covered with a biocompatible insulator. An exemplary biocompatible insulator may be made of at least one of polypyrrole, polylactic acid (PLA), silicone rubber, polyethylene terephthalate (PET), polydimethylsiloxane (PDMS), polyethylene, and combinations thereof. In an exemplary embodiment, fixed needle 302 may be shielded with the biocompatible insulator so that fixed needle 302 may be electrically insulated from undesired objects before reaching cancerous tumor 106, for example, when electrode 300 may have to pass through skin or other organs of a patient's body to reach cancerous tumor 106.

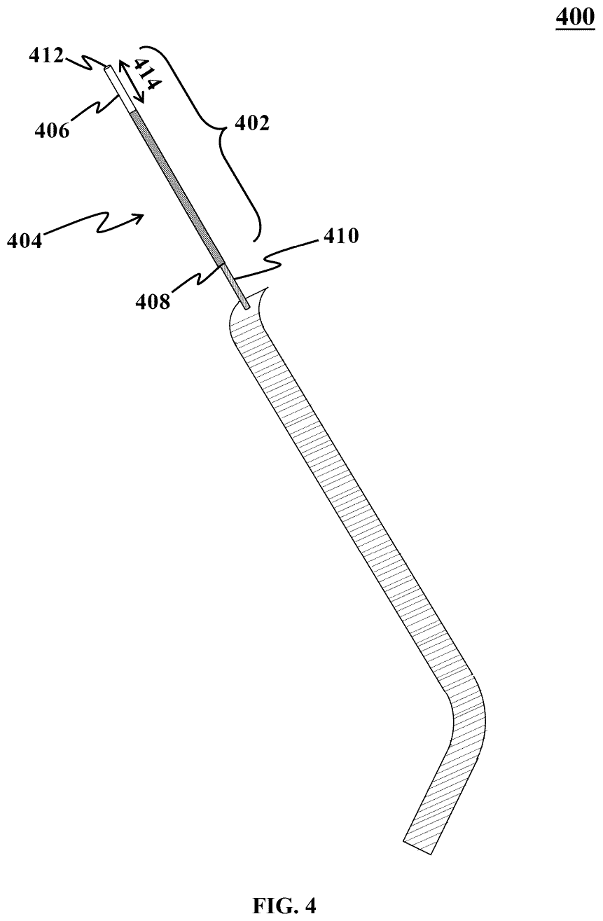

FIG. 4 shows a schematic of an electrode with a replaceable needle partially covered with a biocompatible insulator, consistent with one or more exemplary embodiments of the present disclosure. Referring to FIG. 2, FIG. 3, and FIG. 4, an exemplary electrode 400 may have a similar structure to electrode 300 and may further include an exemplary replaceable needle 402 (similar to replaceable needle 206) that may be partially covered with a biocompatible insulator. An exemplary biocompatible insulator may be made of at least one of polypyrrole, polylactic acid (PLA), silicone rubber, polyethylene terephthalate (PET), polydimethylsiloxane (PDMS), polyethylene, and combinations thereof. In an exemplary embodiment, replaceable needle 402 may include a proximal portion 404 and a distal portion 406. In an exemplary embodiment, proximal portion 404 may be covered with a biocompatible insulator and may allow for being attached to a tip 408 (similar to tip 228) of a fixed needle 410 (similar to fixed needle 302). In an exemplary embodiment, distal portion 406 may include a tip 412 of replaceable needle 402. Referring to FIG. 1 and FIG. 4, in an exemplary embodiment, tip 412 of replaceable needle 402 may allow for being in contact with cancerous tumor 106. An exemplary length 414 of distal portion 406 may be between about 1 cm and about 3 cm. In an exemplary embodiment, proximal portion 404 may be shielded with the biocompatible insulator so that replaceable needle 402 may be electrically insulated from undesired objects before reaching cancerous tumor 106, for example, when electrode 400 may have to pass through skin or other organs of a patient's body to reach cancerous tumor 106. As a result, in an exemplary embodiment, only tip 412 may contact cancerous tumor 106 so that electroporation process may be targeted to an exact position on cancerous tumor 106 determined by an operator of system 100.

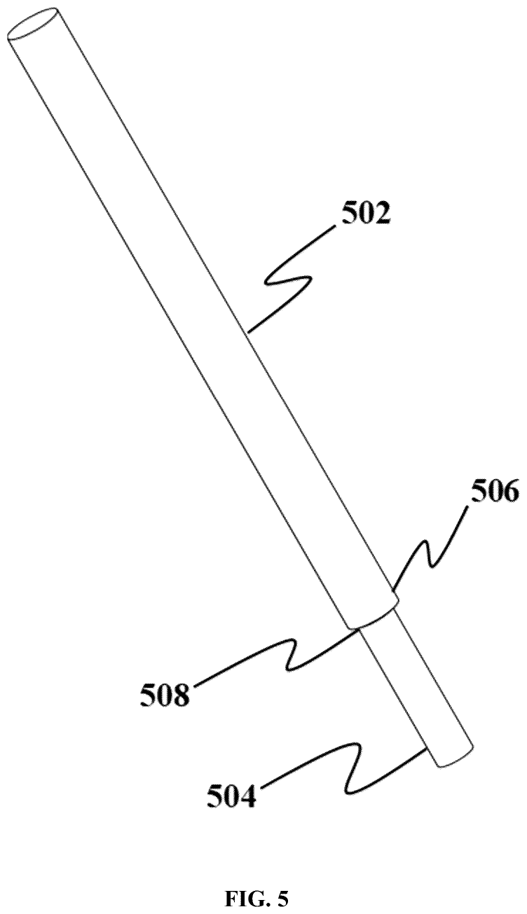

FIG. 5 shows a schematic of replaceable needle attached to a fixed needle, consistent with one or more exemplary embodiments of the present disclosure. Referring to FIG. 2 and FIG. 5, an exemplary replaceable needle 502 may be similar to replaceable needle 206 and an exemplary fixed needle 504 may be similar to fixed needle 204. In an exemplary embodiment, replaceable needle 502 may be a hollow tube. An exemplary proximal end 506 of the hollow tube may encompass a tip 508 (similar to tip 228) of fixed needle 504.

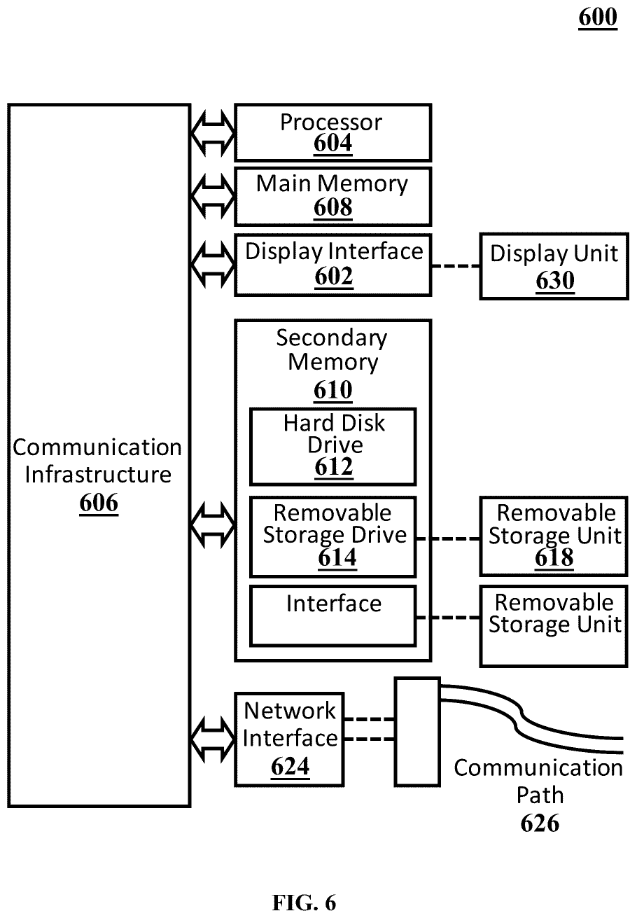

FIG. 6 shows an example computer system 600 in which an embodiment of the present invention, or portions thereof, may be implemented as computer-readable code, consistent with exemplary embodiments of the present disclosure. For example, processor 105 may be implemented in computer system 600 using hardware, software, firmware, tangible computer readable media having instructions stored thereon, or a combination thereof and may be implemented in one or more computer systems or other processing systems. Hardware, software, or any combination of such may embody any of the modules and components in FIG. 1-FIG. 5.

If programmable logic is used, such logic may execute on a commercially available processing platform or a special purpose device. One ordinary skill in the art may appreciate that an embodiment of the disclosed subject matter can be practiced with various computer system configurations, including multi-core multiprocessor systems, minicomputers, mainframe computers, computers linked or clustered with distributed functions, as well as pervasive or miniature computers that may be embedded into virtually any device.

For instance, a computing device having at least one processor device and a memory may be used to implement the above-described embodiments. A processor device may be a single processor, a plurality of processors, or combinations thereof. Processor devices may have one or more processor “cores.”

An embodiment of the invention is described in terms of this example computer system 600. After reading this description, it will become apparent to a person skilled in the relevant art how to implement the invention using other computer systems and/or computer architectures. Although operations may be described as a sequential process, some of the operations may in fact be performed in parallel, concurrently, and/or in a distributed environment, and with program code stored locally or remotely for access by single or multi-processor machines. In addition, in some embodiments the order of operations may be rearranged without departing from the spirit of the disclosed subject matter.

Processor device 604 may be a special purpose (e.g., a graphical processing unit) or a general-purpose processor device. As will be appreciated by persons skilled in the relevant art, processor device 604 may also be a single processor in a multi-core/multiprocessor system, such system operating alone, or in a cluster of computing devices operating in a cluster or server farm. Processor device 604 may be connected to a communication infrastructure 606, for example, a bus, message queue, network, or multi-core message-passing scheme.

In an exemplary embodiment, computer system 600 may include a display interface 602, for example a video connector, to transfer data to a display unit 630, for example, a monitor. Computer system 600 may also include a main memory 608, for example, random access memory (RAM), and may also include a secondary memory 610. Secondary memory 610 may include, for example, a hard disk drive 612 and a removable storage drive 614. Removable storage drive 614 may include a floppy disk drive, a magnetic tape drive, an optical disk drive, a flash memory, or the like. Removable storage drive 614 may read from and/or write to a removable storage unit 618 in a well-known manner. Removable storage unit 618 may include a floppy disk, a magnetic tape, an optical disk, etc., which may be read by and written to by removable storage drive 614. As will be appreciated by persons skilled in the relevant art, removable storage unit 618 may include a computer usable storage medium having stored therein computer software and/or data.

In alternative implementations, secondary memory 610 may include other similar means for allowing computer programs or other instructions to be loaded into computer system 600. Such means may include, for example, a removable storage unit 622 and an interface 620. Examples of such means may include a program cartridge and cartridge interface (such as that found in video game devices), a removable memory chip (such as an EPROM, or PROM) and associated socket, and other removable storage units 622 and interfaces 620 which allow software and data to be transferred from removable storage unit 622 to computer system 600.

Computer system 600 may also include a communications interface 624. Communications interface 624 allows software and data to be transferred between computer system 600 and external devices. Communications interface 624 may include a modem, a network interface (such as an Ethernet card), a communications port, a PCMCIA slot and card, or the like. Software and data transferred via communications interface 624 may be in the form of signals, which may be electronic, electromagnetic, optical, or other signals capable of being received by communications interface 624. These signals may be provided to communications interface 624 via a communications path 626. Communications path 626 carries signals and may be implemented using wire or cable, fiber optics, a phone line, a cellular phone link, an RF link or other communications channels.

In this document, the terms “computer program medium” and “computer usable medium” are used to generally refer to media such as removable storage unit 618, removable storage unit 622, and a hard disk installed in hard disk drive 612. Computer program medium and computer usable medium may also refer to memories, such as main memory 608 and secondary memory 610, which may be memory semiconductors (e.g. DRAMs, etc.).

Computer programs (also called computer control logic) are stored in main memory 608 and/or secondary memory 610. Computer programs may also be received via communications interface 624. Such computer programs, when executed, enable computer system 600 to implement different embodiments of the present disclosure as discussed herein. In particular, the computer programs, when executed, enable processor device 604 to implement the processes of the present disclosure, such as the operations in system 100 illustrated in FIG. 1 discussed above. Accordingly, such computer programs represent controllers of computer system 600. Where an exemplary embodiment of system 100 is implemented using software, the software may be stored in a computer program product and loaded into computer system 600 using removable storage drive 614, interface 620, and hard disk drive 612, or communications interface 624.

Embodiments of the present disclosure also may be directed to computer program products including software stored on any computer useable medium. Such software, when executed in one or more data processing device, causes a data processing device to operate as described herein. An embodiment of the present disclosure may employ any computer useable or readable medium. Examples of computer useable mediums include, but are not limited to, primary storage devices (e.g., any type of random-access memory), secondary storage devices (e.g., hard drives, floppy disks, CD ROMS, ZIP disks, tapes, magnetic storage devices, and optical storage devices, MEMS, nanotechnological storage devices, etc.).

The embodiments have been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications, and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various implementations. This is for purposes of streamlining the disclosure, and is not to be interpreted as reflecting an intention that the claimed implementations require more features than are expressly recited in each claim. Rather, as the following claims reflect, inventive subject matter lies in less than all features of a single disclosed implementation. Thus, the following claims are hereby incorporated into the Detailed Description, with each claim standing on its own as a separately claimed subject matter.

While various implementations have been described, the description is intended to be exemplary, rather than limiting and it will be apparent to those of ordinary skill in the art that many more implementations and implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that any of the features shown and/or discussed in the present disclosure may be implemented together in any suitable combination. Accordingly, the implementations are not to be restricted except in light of the attached claims and their equivalents. Also, various modifications and changes may be made within the scope of the attached claims.

What is claimed is:

1. A system for electroporation of cancerous tumors, the system comprising:
a pair of electrodes configured to deliver a sequence of electric pulses to a cancerous tumor, each respective electrode of the pair of electrodes comprising:
a body made of a conductive material and covered with a biocompatible insulator, the body comprising:
a grip portion configured to be held by an operator of the system;
an insertion portion configured to be inserted into a patient's body; and
a curved portion between the grip portion and the insertion portion;
a fixed needle made of a conductive material and attached to a tip of the insertion portion; and
a replaceable needle made of a conductive material and configured to be attached to a tip of the fixed needle;
an electric pulse generator electrically coupled to the pair of electrodes;
a memory having processor-readable instructions stored therein; and
one or more processors configured to access the memory and execute the processor-readable instructions, which, when executed by the one or more processors configures the electric pulse generator to generate the sequence of electric pulses.

2. The system of claim 1, wherein an angle between an axis of the grip portion and an axis of the insertion portion is between 90 degrees and 120 degrees.

3. The system of claim 1, wherein a length of the insertion portion is two to three times larger than a length of the grip portion.

4. The system of claim 1, wherein the fixed needle is covered with a biocompatible insulator.

5. The system of claim 4, wherein the replaceable needle comprises:
a proximal portion covered with a biocompatible insulator and configured to be attached to the tip of the fixed needle; and
a distal portion comprising a tip of the replaceable needle, the tip of the replaceable needle configured to contact the cancerous tumor.

6. The system of claim 5, wherein a length of the distal portion is between 1 cm and 3 cm.

7. The system of claim 1, wherein the replaceable needle comprises a hollow tube, a proximal end of the hollow tube configured to encompass the tip of the fixed needle.

8. The system of claim 1, wherein the sequence of electric pulses comprises electric pulses with a magnitude in a range of 400 V/cm to 1500 V/cm and a duration in a range of 90 μs to 110 μs.

9. The system of claim 1, wherein each of the body, the fixed needle, and the replaceable needle is made of metal.

10. The system of claim 1, wherein the electric pulse generator comprises a pair of wires, each respective wire of the pair of wires electrically coupled to a respective electrode of the pair of electrodes.

11. The system of claim 1, further comprising a pair of connectors, each respective connector of the pair of connectors configured to connect a respective wire of the pair of wires to a respective electrode of the pair of electrodes.

12. The system of claim 1, further comprising an imaging system configured to capture real-time images from the cancerous tumor, surrounding environment of the cancerous tumor, and a path from a respective location of each of the pair of electrodes to the cancerous tumor.
